(19) United States
(12) Patent Application Publication
(10) Pub. No.: US 2021/0272699 A1
(43) Pub. Date: Sep. 2, 2021

(54) METHOD AND SYSTEM FOR ASSESSING A CORONARY STENOSIS

(71) Applicant: University of Louisville Research Foundation, Inc., Louisville, KY (US)

(72) Inventors: Robert E. Berson, Louisville, KY (US); Javad Hashemi, Louisville, KY (US); Shahab Ghafghazi, Louisville, KY (US)

(21) Appl. No.: 17/261,227

(22) PCT Filed: Jul. 19, 2019
(86) PCT No.: PCT/US2019/042508

§ 371 Date: Jan. 19, 2021

Related U.S. Application Data
(60) Provisional application No. 62/701,136, filed on Jul. 20, 2018.

(51) Int. Cl. G16H 50/50; A61B 5/029; A61B 5/00; G16H 50/30; G06T 17/00; G06T 7/00

(52) U.S. Cl. CPC G16H 50/50; A61B 5/029; A61B 5/7246; G16H 50/30; G06T 2207/10028; G06T 7/0012; A61B 2560/02; G06T 2207/30104; G06T 17/00

(57) ABSTRACT

A non-invasive computer-based method and system for assessing a coronary stenosis or other blockage in an artery or other vasculature includes creating a computational model of the vasculature of interest, modeling blood flow through the vasculature, and determining the mean residence time through a given coronary artery segment, which is a direct assessment of physiological changes on the flow of blood as a result of the stenosis. In some embodiments, blood is modeled as a multi-phase fluid.

This application claims the benefit of U.S. provisional patent application Ser. No. 62/701,136, filed 20 Jul. 2018, for METHOD AND SYSTEM FOR ASSESSING A CORONARY STENOSIS, incorporated herein by reference.

### GOVERNMENT LICENSE RIGHTS

This invention was made with government support under Grant No. 1355438 awarded by the U.S. National Science Foundation and Award No. 5U01HL127518-03 awarded by the U.S. National Institutes of Health. The government has certain rights in the invention.

### FIELD OF THE INVENTION

A non-invasive computer-based method and system for assessing a coronary stenosis or other blockage in an artery or other vasculature includes creating a computational model of the vasculature of interest, modeling blood flow through the vasculature, and determining the mean residence time through a given coronary artery segment, which is a direct assessment of physiological changes on the flow of blood as a result of the stenosis. In some embodiments, blood is modeled as a multi-phase fluid.

### BACKGROUND

The origin of cardiac events, such as myocardial infarction and aneurysm, are attributed to various hemodynamic factors, such as shear stress of regions of stagnant flow within the coronary arteries or other vasculature. As a result, in the U.S., more than one million invasive coronary angiography (ICA) procedures are performed every year in patients who present with chest pain or are known to have stable coronary artery disease (CAD). The goal of the ICA procedure is to determine if there is any significant blockage (stenosis) that limits blood flow to the heart muscle in the coronary arteries. Almost half of ICA procedures culminate in stent placement in coronary arteries in order to relieve the blockage of blood flow. The cardiologist performing the ICA procedure in the cardiac catheterization lab determines the significance of the stenosis by one of two methods: (i) visually estimating the degree of stenosis (“eyeballing” the stenosis), which is the routine practice and is done in the majority of patients, or (ii) by invasively measuring fractional flow reserve (FFR). In this regard, FFR is defined as the ratio of the mean blood pressure downstream of the stenosis divided by the mean blood pressure upstream from the stenosis; in short, it is a measure of pressure differential across the stenosis. Normal FFR is 1 and an FFR<0.8 is considered hemodynamically significant. Invasively-measured FFR (i-FFR) via pressure-wire is considered optimal as it has been demonstrated to both improve patient outcomes and diminish the cost of healthcare. However, i-FFR is only performed in 10-20% of patients because it is invasive, expensive, and time-consuming, and it also requires more radiation and contrast exposure than visual estimation of the stenosis.

As an alternative, efforts have been made to determine FFR though non-invasive methods. For example, a computer system can be configured to receive patient-specific imaging data regarding a geometry of the heart and vasculature of a patient, such that a three-dimensional model can be created that represents at least a portion of the heart and/or vasculature. The computer system is further configured to create a physics-based model relating to a pressure using computational fluid dynamics (CFD), and the computer system can then noninvasively determine a virtual FFR (v-FFR) based on the three-dimensional model and the physics-based model. Specifically, the computer system determines pressure loss across a stenosis or other blockage. See, for example, U.S. Pat. Nos. 8,315,813, 9,189,600, and 9,339,200, and U.S. Patent Publication Nos. 2015/0302139 and 2016/0066861.

Determining v-FFR accurately depends on accuracy of the geometric renderings and model inputs. Empirical resistance boundary conditions at every coronary outlet are typically used but determining accurate values remains a dilemma. Published data reports 6-12% combined false positives and false negatives for v-FFR as compared to FFR. Both FFR and v-FFR are a function of pressure loss, a form of energy loss due to friction between fluid and the walls or between layers of the fluid itself. There are additional significant frictional losses around bends and through constrictions. In blood flow through stenotic arteries, recirculation regions are known to form distal to the stenosis, which present a major source of frictional, and hence, pressure loss. Blood is typically modeled as laminar, although localized regions of turbulence can exist in a recirculation region, and not accounting for the turbulent energy dissipation may reduce the accuracy of the predicted pressure loss. Even if modeled as turbulent, the velocity terms are still generally empirical.

The determination of the v-FFR requires significant computing resources and, in current practice, patient-specific imaging data is typically transmitted from the medical facility to a remote location where the computer system creates the model and determines the v-FFR. Thus, there remains a need for a non-invasive method and system for assessing a coronary stenosis, especially a method and system which can be implemented locally in a cardiac catheterization lab, provides substantially real-time assessments, and generates fewer combined false positives and false negatives than v-FFR.

### SUMMARY

To address these limitations, disclosed herein is a new non-invasive computational based method to detect and assess coronary stenosis without the use of FFR or other determination of blood pressure. Mean age theory provides a computationally efficient method for computing residence time or “age” of fluid, where “age” refers to the amount of time a parcel of fluid resides between two boundaries. The dimensionless metric, Blood_{RT}, is representative of the average time it takes blood to pass through a given arterial segment, and is indicative of the increase in time as compared to the nominal time spent flowing through that segment in the absence of an obstruction. Increase in residence time is due to a small region of recirculatory flow distal to stenosis as elucidated by model-derived pathlines. In some embodiments, blood is modeled as a multi-phase fluid and the mean age of a constituent of blood (e.g., red blood cells) is determined. The method was applied to one hundred coronary arteries from patients who had already undergone the i-FFR measurement for clinical indications. A threshold for Blood_{RT} was determined that statistically correlates to the FFR 0.80 threshold for hemodynamically significant stenosis, and has excellent discrimination in detecting significant from non-significant stenosis compared to the gold standard pressure-wire-determined i-FFR.

It will be appreciated that the various apparatus and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

Embodiments of the invention described herein are described with particular reference to coronary vasculature. In some embodiments, additionally or alternatively, the vasculature is of another organ, and the systems and methods described herein used to evaluate blood flow through such other vasculature.

### BRIEF DESCRIPTION OF THE DRAWINGS

A better understanding of the present invention will be had upon reference to the following description in conjunction with the accompanying drawings.

FIG. 1 is a flow chart illustrating the steps of an exemplary method for assessing a coronary stenosis in accordance with the present invention.

FIG. 2A is a graph of sample hyperemic velocity inlet (m/s) boundary condition.

FIG. 2B is a graph of sample hyperemic pressure outlet (Pa) boundary condition.

FIG. 3A is a depiction of blood flow pathlines (m/s) in a left anterior descending (LAD) artery segment at 0.15 s (left) and 0.70 s (right) of a pulse in a patient without significant stenosis.

FIG. 3B is a depiction of blood flow pathlines (m/s) in a left anterior descending (LAD) artery segment at 0.15 s (left) and 0.70 s (right) of a pulse in a patient with significant stenosis.

FIG. 4 is a depiction of wall shear stress contours in an artery segment in a patient without significant stenosis (panel A, left side) and in a patient with significant stenosis (panel B, right side). The oval corresponds to the region of recirculatory flow.

FIG. 5A is a depiction of mean residence time (s) pathlines in a left anterior descending (LAD) artery segment at 0.15 s (left) and 0.70 s (right) of a pulse in a patient without significant stenosis.

FIG. 5B is a depiction of mean residence time (s) pathlines in a left anterior descending (LAD) artery segment at 0.15 s (left) and 0.70 s (right) of a pulse in a patient with significant stenosis.

FIG. 6A is a graph of mean residence time throughout one cardiac pulse for original outlet pressure, half the original outlet pressure, and 0 outlet pressure in a patient without significant stenosis.

FIG. 6B is a graph of mean residence time throughout one cardiac pulse for original outlet pressure, half the original outlet pressure, and 0 outlet pressure in a patient with significant stenosis.

FIG. 7 is a plot of calculated Blood_{RT} versus i-FFR.

FIG. 8 depicts a receiver operator characteristic (ROC) curve plotting the true positive rate (sensitivity) as a function of the false positive rate (1-specificity) for Blood_{RT}.

### DETAILED DESCRIPTION OF THE PREFERRED EMBODIMENTS

Mean residence time is employed to characterize blood flow characteristics in coronary segments. Parameters such as relative velocity and wall shear stress (WSS) are indicative of changes in flow characteristics, but by themselves do not necessarily correlate to physiologic significance in stenotic coronary arteries. On the other hand, mean residence time or “age” is a widely used established indicator of variance in flow, primarily in industrial systems. Two objects with equal volume and flow rate may have vastly different flow characteristics and, hence, mean residence time values, if their geometries or, in this case, anatomies differ.

One or more of the steps or logic described herein may be implemented using, among other things, a tangible computer-readable storage medium comprising computer-executable instructions (e.g., software code). Alternatively, the steps or logic may be implemented as software code, firmware code, hardware, and/or combination thereof. For example, the steps or logic may be implemented as part of a medical imaging system or otherwise implemented locally in a cardiac catheterization lab. General purpose and dedicated computing devices, standalone or connected (e.g., via a network) to other computing devices, for executing computer-executable instructions generally include a processor, a memory, input/output circuits, and optionally, non-transient storage media. The processor communicates with the memory and the input/output circuits via one or more buses. The input/output circuits can be used to transfer information between the memory and other computer systems or a network using, for example, an Internet Protocol (IP) connection, wired connection or wireless connection. These components may be conventional components as are generally known in the art.

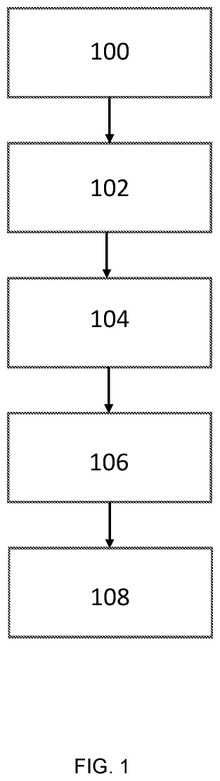

Referring now to FIG. 1, in this exemplary implementation, the first step is to obtain at least one anatomical image of a vasculature of interest from a patient, as indicated by block 100 of FIG. 1. To test this invention, one hundred arteries from ninety patients who had undergone coronary angiography and FFR measurements for clinical indications were included in this study. Patients' characteristics are detailed in Table 1 below:

TABLE 1
Clinical Characteristics of Patients

| | |
|---|---|
| Total Patients | 90 |
| Total vessels | 100 |
| Age | 63.3 ± 27.6 |
| Male Gender | 57 (63.3%) |
| Hypertension | 87 (96.6%) |
| Diabetes mellitus | 40 (44.4%) |
| Current smoker (last 1 year) | 34 (37.7%) |
| History of prior myocardial | 35 (38.9%) |
| infarction | |
| History of prior PCI | 42 (46.6%) |
| History of prior CAD | 57 (63.3%) |
| Hyperlipidemia | 74 (82.2%) |
| Family history | 29 (32.2%) |
| Vessel disease: | |
| Single-vessel | 83 (83%) |
| Two-vessel | 4 (4%) |
| Three-vessel | 3 (3%) |
| Total vessels | 100 |

Patients with stenosis in a major epicardial artery (left anterior descending artery [LAD], left circumflex [LCx]/obtuse marginal [OM] and right coronary artery [RCA]) were eligible for inclusion in the study. Exclusion criteria were: significant left main disease, coronary arteries with bifurcational lesions, and coronary arteries distally protected by bypass grafts. All lesions included in the study had documented adenosine administration and i-FFR recording, as well as suitable angiographic projections for three-dimensional (3D) reconstruction.

Referring again to FIG. 1, the second step is to create a three-dimensional model of the vasculature of interest from such images, as indicated by block 102 of FIG. 1. There are well-known technologies and commercially available products for achieving these first two steps. For instance, the images can be obtained by angiogram, computerized tomography (CT) scan, or any similar imaging means. Then, commercially available software tools can be used to create the three-dimensional model from such images.

In one exemplary implementation, at least two two-dimensional angiographic images are obtained of the vasculature in the area of a stenosis; such images are obtained from different angles (e.g., two images separated by 30°). The images are then input into a commercial software package, such as the CAAS 7.5 QCA-3D system (Pie Medical Imaging, Maastricht, The Netherlands), and the output is a 3D model of the vasculature. Another commercial software package, syngo IZ3D, which is available from Siemens Healthcare GmbH of Erlangen, Germany, may also be suitable for creating the three-dimensional model.

Referring again to FIG. 1, once the 3D model has been created, the next step is to apply CFD principles to develop a model of blood flow through the vasculature, as indicated by block 104 of FIG. 1. There are known technologies and commercially available products for developing the model of blood flow. For instance, in one exemplary implementation, the model is developed using ANSYS Fluent v.17.1 CFD software, which is available from ANSYS, Inc. of Canonsburg, Pa. In some embodiments, the blood flow modeling software is hosted on the same computing device as the 3D modeling software. Reynolds numbers were between 128-1501 in the region of stenosis, so flow was modeled as laminar. Blood viscosity, a characteristic for hemodynamic flow modeling, was modeled according to Newtonian viscosity using each patient's measured viscosity. Unstructured computational meshes were built as tetrahedral shaped cells using ANSYS Mesher 17.0. An optimal node count of 542,000 was determined by mesh sensitivity analysis of mean residence time for an artery with volume of 4.04×10⁻⁸ m³, and then scaled accordingly for the size of each case.

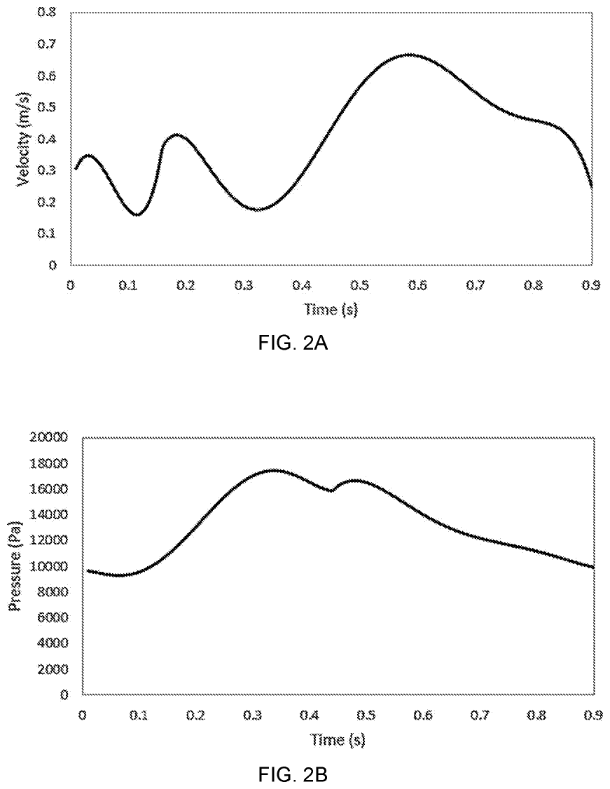

The inlet boundary condition was a transient velocity waveform (FIG. 2A) representing the coronary blood cycle. The outlet boundary condition was a pressure waveform (FIG. 2B). Both were scaled to match the mean flow in hyperemic conditions and pressure measured for each patient, and then programmed using user defined functions (UDF). Similar to node count, a sensitivity analysis determined an optimal time step size of 0.01 s.

Once the three-dimensional model of the vasculature of interest has been created and the CFD principles have been applied to model blood flow through the vasculature, a determination is made as to the mean residence time (or “mean age”) of blood traveling through the vasculature of interest (i.e., through the region of restricted flow) from a first position to a second position, as indicated by block 106 of FIG. 1. The determination is preferably made via software hosted on a computing device, such as the computing device described above. The first position is proximal or upstream of the location of the possible stenosis and the second position is distal or downstream of the location of the possible stenosis. In further embodiments, a determination is made as to the ratio of nominal mean residence time (i.e., the volume of the vascular segment divided by the flow rate) to the determined mean residence time, as also indicated by block 106 of FIG. 1. This dimensionless value is termed Blood_{RT}.

Referring again to FIG. 1, as a final step in block 108, the determined mean residence time of the blood travelling through the vasculature of interest from the first position to the second position, as represented by Blood_{RT}, can be correlated to a severity of the stenosis. The correlation of mean residence time and Blood_{RT} to stenosis severity is discussed in further detail below.

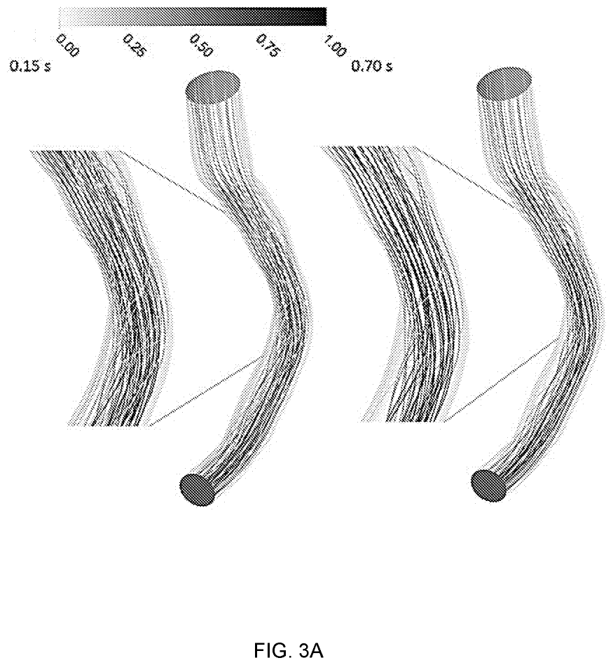

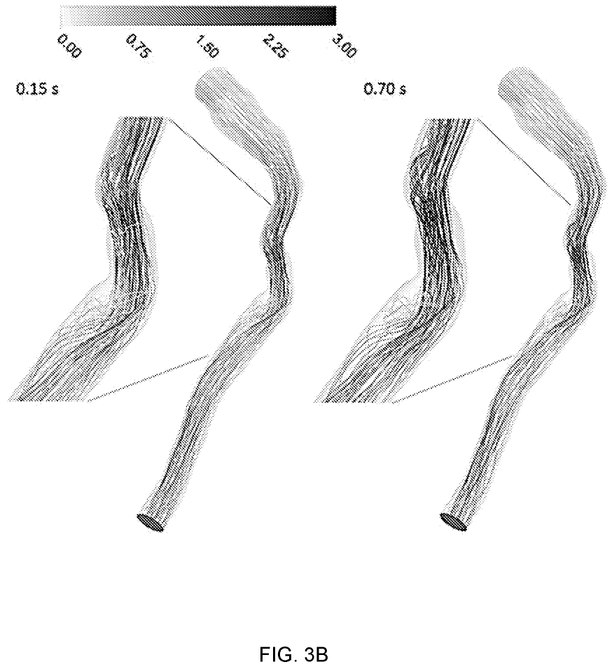

Blood flow pathlines are shown in two left anterior descending (LAD) artery segments as representative examples of one case above and one below the FFR threshold (FIGS. 3A and 3B, respectively). Patient A, shown in FIG. 3A, had a non-significant stenosis with FFR equal to 0.94. Patient B, shown in FIG. 3B had a significant stenosis with FFR equal to 0.63. Pathlines remain relatively ordered for Patient A during both systole (at 0.15 s of the pulse) and diastole (at 0.70 s of the pulse), while pathlines reveal a small but noticeable region of low velocity recirculation and holdup distal to the stenosis for Patient B, especially during diastole. In Patient A, the maximum velocity during diastole was only about ˜40% greater than the inlet velocity (˜1.0 m/s compared to ˜0.72 m/s) at this point in the pulse input (FIG. 2A shows the velocity input pulse for Patient A), while for Patient B the maximum velocity was about 650% greater than the inlet velocity at this point (˜3 m/s compared to ˜0.4 m/s) in the pulse (pulse not shown).

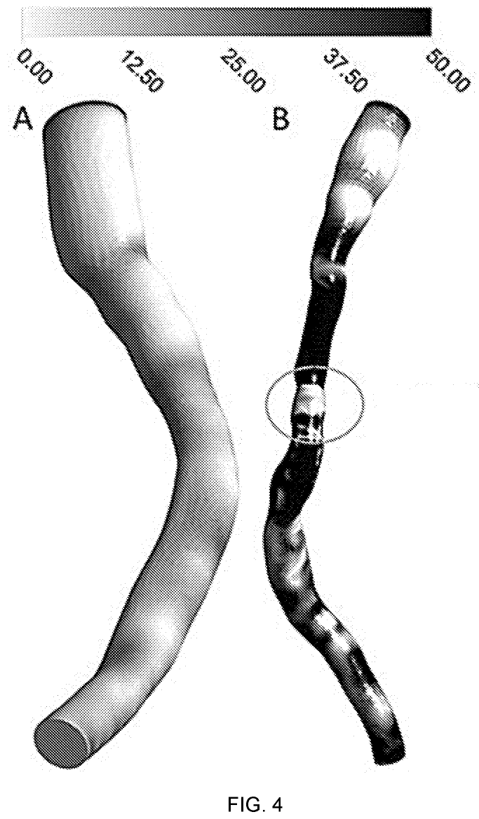

Referring now to FIG. 4, panel B, Patient B has an elongated stenosis with high WSS throughout the stenosed region, but with a noticeable region of low WSS corresponding to the area of recirculation, as indicated by the oval in the figure. Referring now to panel A, WSS is generally more ordered with little variability for Patient A. Both images are during systole, at 0.7 s of the pulse.

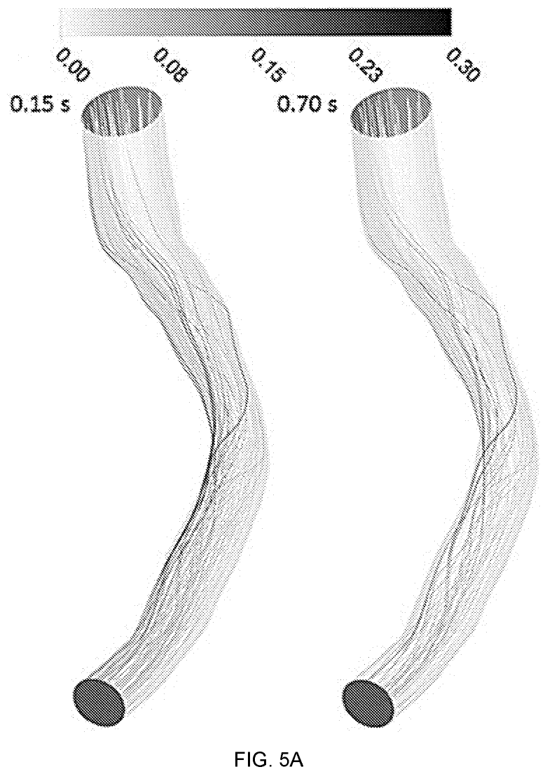

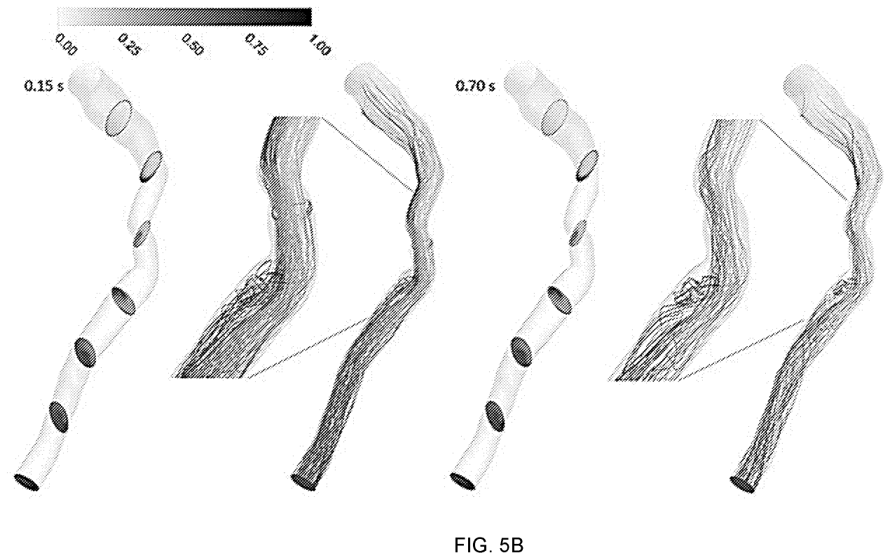

FIGS. 5A and 5B show pathlines colored by mean residence time for Patients A and B, respectively. The color in FIG. 5A indicates a methodical increase in residence time from inlet to outlet for Patient A since there is no significant obstacle to blood flow in this healthy patient. The overall mean residence time was 0.0817 s, just 22% above its nominal mean residence time of 0.0670 s, where nominal mean residence time is defined as volume divided by flow rate and represents the mean residence time that would be expected if flow was completely uninhibited. However for Patient B, as shown in FIG. 5B, mean residence time in the recirculation region distal to stenosis is high relative to the fluid passing in the main jet stream. The bulk of the mean residence time in this region is approximately 50% higher than the main jet stream, with certain points being 3×-4× higher. The overall mean residence time for this patient is 0.0796 s, while its nominal mean residence time was 0.0535 s, an increase of 49%.

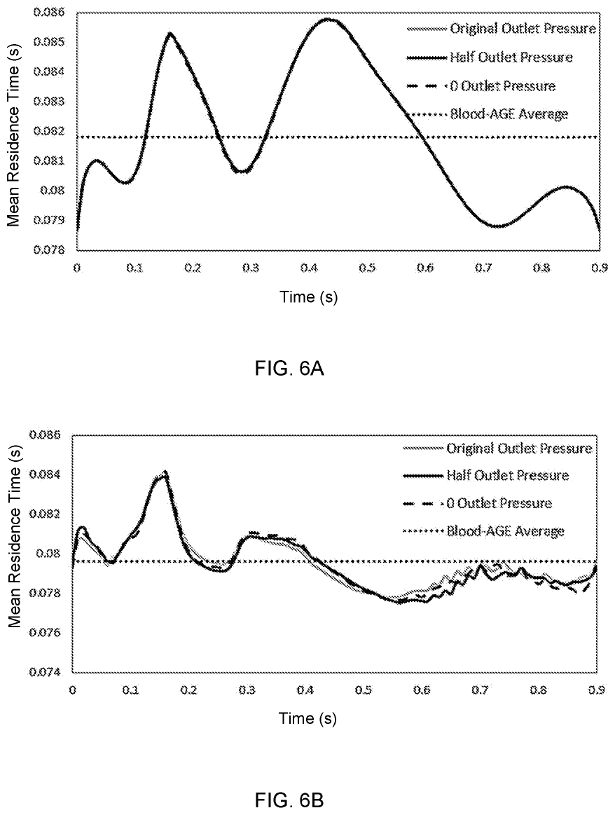

Under normal conditions, mean residence time should increase during the systolic phase, when the velocity is generally lower, and decrease in the diastolic phase, when the velocity is generally higher. As shown in FIG. 6A, mean residence time for Patient A over the course of an entire pulse reflects this, where the amplitudes are low when velocity amplitudes are high and vice-versa. Also, as the slope of the velocity is increasing, the slope of mean residence time is decreasing and vice-versa. As shown in FIG. 6B, mean residence time adheres to this pattern during systole for Patient B, but generally levels off during diastole when it should be decreasing, which is reflective of the recirculation and hold-up of blood flow during the diastolic phase. The mean residence time in FIGS. 6A and 6B represents the mean exit residence time of blood that entered the arterial segment at a given time during the cardiac cycle, and the overall mean value is reported as the average of mean residence times over one complete cycle.

The pressure outlet boundary condition did not affect mean age for Patient A (FIG. 6A) or Patient B (FIG. 6B). FIG. 6A shows age throughout one pulse for the original pressure, half the original pressure, and zero (gauge) pressure for Patient A. Mean age for these three examples were 0.0818±0.00001 s. FIG. 6B shows the same for Patient B with a mean age of 0.0796±0.00009 s.

As noted above, Blood_{RT} is defined as a dimensionless age parameter to account for varying length and volume of each arterial segment plus varying blood flow rates, such that (Blood_{RT}=Nominal Mean Residence Time(s)/Mean Residence Time(s)). Mean residence time was first determined in 100 coronary arteries for which the i-FFR was known.

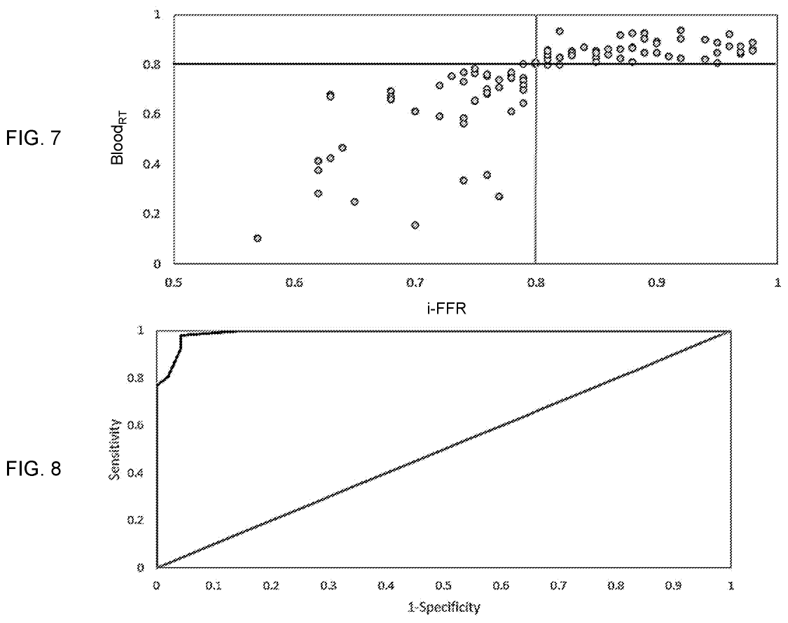

The correlation between Blood_{RT} and i-FFR was studied using the Pearson (r) correlation coefficient. Observations are grouped into two groups, abnormal pressure-wire FFR (<=0.80) and normal i-FFR (>0.80). There is a strong correlation between pressure-wire FFR and Blood_{RT} (r=0.75, P<0.001). Abnormal (FFR≤1.80) and normal (FFR>0.80) groups based on the i-FFR cutoff are highly associated with a Blood_{RT} cut off 0.80. There were 46 true negatives (46%), 51 true positives (51%), 1 false negative (1%) and, 2 false positives (2%) (FIG. 7). The sensitivity and specificity of Blood_{RT}, along their 95% confidence intervals, are 98% (88-100) and 96% (86-100) respectively, indicating strong ability for Blood_{RT} to predict whether FFR is above or below 0.80. These AUC, sensitivity, and specificity values compared favorably to various forms of v-FFR, as shown in Table 2 below:

TABLE 2
Statistical analysis comparison between Blood_{RT}
and various forms of virtual FFR

| | Case | | | |
|---|---|---|---|---|
| Metrics | Numbers | AUC | Sensitivity | Specificity |
| Blood_{RT} | 100 | 0.996 | 96 | 98 |
| FFR_{angio} | 184 | 0.97 | 88 | 95 |
| QFR | 87 | 0.91 | 78 | 89 |
| FFR_{QCA} | 77 | 0.93 | 78 | 93 |
| vFAI | 139 | 0.92 | 90.4 | 86.2 |
| Virtual FFR-VIRTU- | 35 | | 71 | 100 |
| 1 | | | | |
| Stenosis flow | 110 | | 93 | 85 |
| reserve (SFR) | | | | |

Receiver operator characteristic (ROC) curve analysis was performed, as shown in FIG. 8.

Pressure-wire FFR, typically considered the gold standard for diagnosing the physiological significance of coronary stenosis, is a function of pressure loss across the stenotic segment. Pressure loss is a characterization of the energy loss in the blood flow resulting from the altered course of flow due to stenosis. The altered, disordered flow leads to frictional loss between layers of fluid, fluid and the wall, and especially around bends and through constrictions, resulting in loss of pressure. Instead of measuring (i-FFR) or computing (v-FFR) pressure loss to quantify the physiological significance of stenosis, the present invention uses a novel approach to quantify altered flow trajectories via the residence time metric, arguably a more direct measure of altered blood flow due to stenosis.

Stenotic flows exhibit flow separation downstream of the stenosis characterized by a central jet stream and secondary flow near the wall, with a strong shear layer in between. The deceleration of flow during diastole is responsible for the conditions that create the secondary flow reversal downstream of the stenosis. The flow separation depends on the upstream flow velocity and diameter of the stenosis. The velocity gradient and shear layer at the interface provide the potential for reversed flow due to the tangential force. This effect occurred here just past the region of stenosis as shown in FIG. 3B.

Mean residence time increased relative to nominal mean residence time due to flow characteristics distal to the stenosis zone, with practically no effect on mean residence time proximal to the stenosis. Even a small fraction of blood held up while recirculating in the secondary flow region will cause the overall mean residence time to increase above the nominal mean residence time value. Higher mean residence time in the recirculation region associated with Patient B was on the order of 1.5×-4× the surrounding fluid that passes uninhibited, contributing to the overall increase in mean age at the exit or, by definition, decrease in the dimensionless Blood_{RT}. Blood_{RT} for patient B, the unhealthy patient with a LAD i-FFR=0.63, was 0.67. Both values indicate an extreme departure from their respective thresholds and are representative of severely disturbed flow due to an elongated stenosis.

While the recirculation pattern generally remains consistent over time, fluid that enters this region eventually crosses back into the primary flow stream at the boundary between the primary and secondary streams. Otherwise, if even a small amount of fluid were held up there indefinitely, mean residence time would approach infinity. The hold-up time and variance from nominal mean residence time depends on the combination and interactions of factors such as velocity through the stenosed area, the size of the stenosis, and shape of the artery segment such as if it is straight or bends.

The threshold between a hemodynamically significant or non-significant stenosis was determined for this novel Blood_{RT} metric, and was determined based on statistical correlation with i-FFR. Blood_{RT} agreed with i-FFR in all but three cases on the hemodynamic significance of the stenosis and decision to stent or not. It is noteworthy that the non-compliant cases also were within ˜0.5% of the statistically determined threshold; the Blood_{RT} of the two false positives were 0.796 and 0.797, and the Blood_{RT} of the false negative was 0.802. Both the Blood_{RT} and FFR thresholds equal to a dimensionless value of ˜0.80. Blood_{RT} is a measure of relative time while FFR is a measure of relative pressure. The two are indirectly related through fluid flow phenomena, but there is no reason other than coincidence that the two should be equal. It is possible that the Blood_{RT} threshold may shift as more cases are studied, but given the strong statistical correlation, any shift would likely be minimal. The similarity in thresholds does not imply that values should correlate for individual cases, however there was a close correlation between Blood_{RT} and FFR (r=0.753, p<0.0001). Patient B provides a sound example with i-FFR=0.62 and Blood_{RT}=0.67.

In embodiments of the present invention, blood may be modeled as a single phase fluid, as described above, or as a multi-phase fluid, which allows for the modeling and tracking of each physical phase (e.g., red blood cells, white blood cells, platelets, and liquid plasma) independently from each other.

With respect to the development of a model of blood flow through the vasculature, in one exemplary implementation, multi-phase mean age (MMA) theory is used to develop the model of blood flow through the vasculature and then determine the mean residence time of red blood cells (RBCs). The use of MMA theory is described in detail in David Chandler Russ, Robert Eric Berson, “Mean age theory in multiphase systems,” Chemical Engineering Science, Volume 141, 17 Feb. 2016, Pages 1-7, which explains that mean age theory as a means of modeling the time dependent behavior of a passive scalar in a steady-state CFD simulation in a multi-phase system begins with the assumption that C(x,t) is the concentration of the scalar tracer at a given location x and time t, without further definition. Here, C(x,t) is defined as:

C(x,t)=ρ·φ(x,t) (Eq. 1)

where ρ is the density of the single phase and φ(x,t) is the scalar value at a given location x and time t. The concentration of a passive scalar confined to a single phase in a multi-phase system can then be defined:

C(x,t)=ρ·α(x,t)·φ(x,t) (Eq. 2)

where α(x, t) is the individual phase volume fraction at a local position and time and ρ is the density of the individual phase. With this definition of scalar concentration for multi-phase systems, the rest of the derivation proceeds analogously to that for a single phase system.

Mean residence time for either definition of C can be defined as:

$$\overline{t} = \frac{{\int }_{0}^{∞} t {C}_{out} dt}{{\int }_{0}^{∞} {C}_{out} dt} \qquad ( Eq . 3 )$$

and can then be generalized to any point in the system by defining “mean residence time” as:

$$a ( x ) = \frac{{\int }_{0}^{∞} tC ( x , t ) dt}{{\int }_{0}^{∞} C ( x , t ) dt} \qquad ( Eq . 4 )$$

This can be solved for any given point in the system. To do so, one must begin with the transient passive scalar advection-diffusion transport equation:

$$\frac{\partial C}{\partial t} + ∇ \cdot ( uC ) = ∇ \cdot ( D ∇ C ) \qquad ( Eq . 5 )$$

Multiplying both sides by time t and integrating yields:

$${\int }_{0}^{∞} t \frac{\partial C}{\partial t} dt + {\int }_{0}^{∞} ∇ \cdot ( tuC ) dt = {\int }_{0}^{∞} ∇ \cdot D ∇ ( tC ) dt \qquad ( Eq . 6 )$$

The first term on the left can be integrated by parts to give:

$${\int }_{0}^{∞} t \frac{\partial C}{\partial t} dt = tC {\vert}_{0}^{∞} - {\int }_{0}^{∞} Cdt \qquad ( Eq . 7 )$$

Since for a pulse input in an open system it is known that:

$${lim}_{t \to ∞} tc = 0 \qquad ( Eq . 8 )$$

It can be inferred that:

$${\int }_{0}^{∞} t \frac{\partial C}{\partial t} dt = - {\int }_{0}^{∞} Cdt \qquad ( Eq . 9 )$$

Taking Eq. 9 and substituting it back into Eq. 6 gives:

$$- 1 + ∇ \cdot \{ u [ \frac{{\int }_{0}^{∞} tCdt}{{\int }_{0}^{∞} Cdt} ] \} = V \cdot \{ D [ \frac{{\int }_{0}^{∞} t C d t}{{\int }_{0}^{∞} Cdt} ] \} \qquad ( Eq . 10 )$$

Finally, substituting in Eq. 4 generates the age transport equation:

∇·(ua)=∇·D∇a+1 (Eq. 11)

In some embodiments where blood is modeled as a multi-phase fluid, a determination is made as to the mean residence time of RBCs travelling through the vasculature of interest from a first position to a second position. Also, a determination is made as to the ratio of nominal mean residence time for RBCs to the determined mean residence time of RBCs, the ratio being designated RBC_{RT}. In initial testing, the mean residence time of RBCs and RBC_{RT} differ from the mean residence time of blood modeled as a single phase fluid and Blood_{RT}, respectively, by only 1% to 2%. As such, single-phase and multi-phase metrics both correlate strongly with stenosis severity.

While discussion of modeling blood flow as a multi-phase fluid is primarily focused on RBCs, it should be understood that multiple physical phases (e.g., red blood cells, white blood cells, platelets, and liquid plasma) of the blood may be modeled and tracked. Furthermore, other methods besides MMA may be used to determine the mean residence time of RBCs and the RBC_{RT}.

Various aspects of different embodiments of the present disclosure are expressed in paragraphs X1, X2, and X3 as follows:

X1: One embodiment of the present disclosure includes a method for assessing a stenosis in a vasculature of interest, comprising the steps of receiving at least one anatomical image including the vasculature of interest; creating a model of the vasculature of interest from the at least one anatomical image; creating a model of blood flow through the vasculature of interest based on the model of the vasculature of interest; and determining a mean residence time of blood travelling through the vasculature of interest from a first position to a second position based on the model of blood flow.

X2: Another embodiment of the present disclosure includes a non-transitory computer readable storage medium storing computer program instructions for assessing a stenosis in a vasculature of interest from anatomical image data, the computer program instructions when executed by a processor cause the processor to perform operations comprising creating a model of the vasculature of interest from the anatomical image data; creating a model of blood flow through the vasculature of interest based on the model of the vasculature of interest; determining a mean residence time of blood travelling through the vasculature of interest from a first position to a second position based on the model of blood flow; and correlating the determined mean residence time to a severity of stenosis.

X3: A further embodiment of the present disclosure includes a computer-implemented method for determining the hemodynamic significance of a stenosis, the method comprising: generating, using a processor, an anatomical model of a vasculature of interest derived from at least one anatomical image; generating, using the processor, a model of blood flow through the vasculature of interest derived from the anatomical model; computing, using the processor, a mean residence time of blood travelling through the vasculature of interest from a first position to a second position derived from the model of blood flow.

Yet other embodiments include the features described in any of the previous paragraphs X1 or X2 or X3 as combined with one or more of the following aspects:

Wherein the at least one anatomical image is a plurality of anatomical images.

Wherein the plurality of anatomical images include two-dimensional angiographic images each including the vasculature of interest, wherein the plurality of two-dimensional angiographic images are obtained from at least two different angles.

Wherein the anatomical images are two two-dimensional angiographic images obtained from two different angles separated by 30 degrees.

Wherein the anatomical image data includes at least two two-dimensional angiographic images including the vasculature of interest, wherein the at least two two-dimensional angiographic images are obtained from different angles.

Wherein the anatomical image data includes two two-dimensional angiographic images obtained from two different angles separated by 30 degrees.

Wherein the method or operation further comprises correlating the determined mean residence time to a severity of stenosis.

Wherein the method of operation further comprises designating the stenosis as hemodynamically significant if the determined mean residence time is less than a predetermined value.

Wherein the predetermined value is about 0.8.

Wherein creating a model of blood flow includes modeling blood as a single-phase fluid or a multi-phase fluid.

Wherein creating a model of blood flow includes modeling blood as a multi-phase fluid.

Wherein creating a model of blood flow includes modeling blood as a multi-phase fluid, the multi-phase fluid including at least red blood cells.

Wherein determining a mean residence time of blood travelling through the vasculature of interest includes determining a mean residence time of red blood cells travelling through the vasculature of interest.

Wherein the method or operation further comprises designating a ratio of nominal mean residence time of red blood cells travelling through the vasculature of interest to the determined mean residence time of red blood cells travelling through the vasculature of interest, and correlating the ratio to a severity of stenosis.

Wherein the method or operation further comprises designating a ratio of nominal mean residence time of blood travelling through the vasculature of interest to the determined mean residence time of blood travelling through the vasculature of interest, and correlating the ratio to a severity of stenosis.

Wherein the first position is proximal to the stenosis and wherein the second position is distal to the stenosis.

Wherein the model of the vasculature of interest is a three-dimensional model.

Wherein correlating the determined mean residence time to a severity of stenosis includes designating a ratio of nominal mean residence time of blood travelling through the vasculature of interest to the determined mean residence time of blood travelling through the vasculature of interest, and designating the stenosis as hemodynamically significant if the ratio is less than a predetermined value.

The foregoing detailed description is given primarily for clearness of understanding and no unnecessary limitations are to be understood therefrom for modifications can be made by those skilled in the art upon reading this disclosure and may be made without departing from the spirit of the invention.

What is claimed is:

1. A method for assessing a stenosis in a vasculature of interest, comprising the steps of:
receiving at least one anatomical image including the vasculature of interest;
creating a model of the vasculature of interest from the at least one anatomical image;
creating a model of blood flow through the vasculature of interest based on the model of the vasculature of interest; and
determining a mean residence time of blood travelling through the vasculature of interest from a first position to a second position based on the model of blood flow.

2. The method of claim 1, wherein the at least one anatomical image is a plurality of anatomical images.

3. The method of claim 2, wherein the plurality of anatomical images include two-dimensional angiographic images each including the vasculature of interest, wherein the plurality of two-dimensional angiographic images are obtained from at least two different angles.

4. The method of claim 1, further comprising correlating the determined mean residence time to a severity of stenosis.

5. The method of claim 1, further comprising designating the stenosis as hemodynamically significant if the determined mean residence time is less than a predetermined value.

6. The method of claim 1, wherein creating a model of blood flow includes modeling blood as a single-phase fluid or a multi-phase fluid.

7. The method of claim 1, wherein creating a model of blood flow includes modeling blood as a multi-phase fluid, the multi-phase fluid including at least red blood cells.

8. The method of claim 7, wherein determining a mean residence time of blood travelling through the vasculature of interest includes determining a mean residence time of red blood cells travelling through the vasculature of interest.

9. The method of claim 7, further comprising:
designating a ratio of nominal mean residence time of red blood cells travelling through the vasculature of interest to the determined mean residence time of red blood cells travelling through the vasculature of interest; and
correlating the ratio to a severity of stenosis.

10. The method of claim 1, further comprising:
designating a ratio of nominal mean residence time of blood travelling through the vasculature of interest to the determined mean residence time of blood travelling through the vasculature of interest; and
correlating the ratio to a severity of stenosis.

11. The method of claim 1, wherein the first position is proximal to the stenosis and wherein the second position is distal to the stenosis.

12. The method of claim 1, wherein the model of the vasculature of interest is a three-dimensional model.

13. A non-transitory computer readable storage medium storing computer program instructions for assessing a stenosis in a vasculature of interest from anatomical image data, the computer program instructions when executed by a processor cause the processor to perform operations comprising:
creating a model of the vasculature of interest from the anatomical image data;
creating a model of blood flow through the vasculature of interest based on the model of the vasculature of interest;
determining a mean residence time of blood travelling through the vasculature of interest from a first position to a second position based on the model of blood flow; and
correlating the determined mean residence time to a severity of stenosis.

14. The non-transitory computer readable storage medium of claim 13, wherein correlating the determined mean residence time to a severity of stenosis includes:
designating a ratio of nominal mean residence time of blood travelling through the vasculature of interest to the determined mean residence time of blood travelling through the vasculature of interest; and
designating the stenosis as hemodynamically significant if the ratio is less than a predetermined value.

15. The non-transitory computer readable storage medium of claim 13, wherein creating a model of blood flow includes modeling blood as a single-phase fluid or a multi-phase fluid.

16. The non-transitory computer readable storage medium of claim 15, wherein creating a model of blood flow includes modeling blood as a multi-phase fluid, the multi-phase fluid including at least red blood cells.

17. The non-transitory computer readable storage medium of claim 13, wherein the first position is proximal to the stenosis and wherein the second position is distal to the stenosis.

18. The non-transitory computer readable storage medium of claim 13, wherein the anatomical image data includes at least two two-dimensional angiographic images including the vasculature of interest, wherein the at least two two-dimensional angiographic images are obtained from different angles.

19. A computer-implemented method for determining the hemodynamic significance of a stenosis, the method comprising:
generating, using a processor, an anatomical model of a vasculature of interest derived from at least one anatomical image;
generating, using the processor, a model of blood flow through the vasculature of interest derived from the anatomical model;
computing, using the processor, a mean residence time of blood travelling through the vasculature of interest from a first position to a second position derived from the model of blood flow.

20. The computer-implemented method of claim 19, further comprising
designating a ratio of nominal mean residence time of blood travelling through the vasculature of interest to the computed mean residence time of blood travelling through the vasculature of interest, and
designating the stenosis as hemodynamically significant if the ratio is less than a predetermined value.
